(19) United States
(12) Patent Application Publication
(10) Pub. No.: US 2015/0148795 A1
(43) Pub. Date: May 28, 2015

(54) RADIO FREQUENCY ABLATION COIL

(71) Applicant: Boston Scientific Scimed, Inc., Maple Grove, MN (US)

(72) Inventors: Michael Devon AMOS, Ayer, MA (US); Bryan Bannon, Duxbury, MA (US); Gary Trendel, Hopkinton, MA (US); Brian Gaffney, Rutland, MA (US); Brad M. Isaacson, Lancaster, MA (US); Anthony Borrelli, Natick, MA (US)

(73) Assignee: Boston Scientific Scimed, Inc., Maple Grove, MN (US)

(21) Appl. No.: 14/551,981

(22) Filed: Nov. 24, 2014

Related U.S. Application Data
(60) Provisional application No. 61/908,935, filed on Nov. 26, 2013.

(51) Int. Cl. A61B 18/14; A61B 10/00; A61B 10/02

(52) U.S. Cl. CPC A61B 18/1477; A61B 10/0233; A61B 10/0045; A61B 2018/00577
USPC 606/41

(57) ABSTRACT

A tumor ablation device may be combined with a diagnostic tool and/or a surgical instrument for offering cancer treatment therapy.

### FIELD OF THE INVENTION

In various embodiments, the present invention relates generally to medical devices having the ability to treat cancerous tissues using radio-frequency ablation coils.

### BACKGROUND

Diagnostic tools, such as endoscopes, are instruments used to examine the interior of a hollow organ or a cavity of the body in order to minimize invasive surgeries. For example, a gastrointestinal (GI) endoscope and a cystoscope can be used to visualize a patient's GI tract and urinary tract to diagnose, respectively, an unexplained diarrhea or hematuria. Endoscopes are generally used to assist in examination of interior body regions; actually treating a disease may requireseparate procedure.

Conventionally, endoscopes may be combined with a variety of surgical instruments to enable surgeons to perform various procedures. For example, a combination of a GI endoscope with an endoscopic retrograde cholangiopancreatography (ERCP) device (e.g., a catheter or an endoscopic ultrasound (EUS) fine needle aspiration (FNA) needle) may be used to treat problems associated with bile and pancreatic ducts. The surgeons may perform procedures, such as opening blocked ducts, draining a tumor cyst, or inserting stents, while imaging the region of interest using video, ultrasound, lasers, optics or other imaging modalities. Typically, treating a benign or malignant tumor cyst requires a separate procedure. As a result, patients must often make repeated clinic visits with attendant inconvenience and delays and, potentially, complications associated with progressing symptoms.

EUS guided FNA (EUS-FNA) has recently been used to drain cystic lesions and collect biopsy samples from them, particulary in the pancreas and the lung, but also in the esophagus and elsewhere. However, current methods for ablating cysts after draining and/or biopsy by EUS-FNA utilize fluid ablative agents such as alcohol or mechanical tools for resection or curettage. These are imprecise and may undesirably spare potentially malignant cystic tissue and/or ablate healthy tissue near the lesion, and a significant need exists in the field for systems and methods which permit more precise tissue ablation in connection with EUS-FNA.

### SUMMARY

In various embodiments, the present invention combines endoscopic imaging and/or surgical instruments, such as endoscopic retrograde cholangiopancreatography (ERCP) devices, with systems and methods for delivering energy to tissue; this allows physicians to image the target region and deliver radiofrequency energy for heating and/or ablation of a portion of the target region immediately, if necessary. In some embodiments, the endoscope and/or ERCP device is combined with an ablation device having a flexible coil electrode at the end of a long metallic core, which electrode is deployable within a benign or malignant tumor cyst. The coil electrode assumed a coiled shape when deposited from a distal end of the device into the cyst cavity to cover the possible greatest amount of surface area therewithin. A radiofrequency source then delivers radiofrequency energy to the target cyst via the coil thereof in an amount sufficient to ablate at least a portion of the cyst or, alternatively, in an amount sufficient to heat the cyst to remodel and/or prevent its continued growth. In one implementation, the coil electrode is combined with an EUS FNA needle to allow physicians to treat the tumor cyst immediately after drainage and/or biopsy of the cyst. The combined system thus permits simultaneously assessing and treating the cancerous tissue, thereby reducing anxiety, complications or side effects associated with typical endoscopic examinations or ERCP procedures. Additionally, devices in accordance with the current invention are advantageously inexpensive and easy to operate.

In one aspect, the present invention relates to an ablation device insertable into a target tissue that includes a core probe, a coil at the distal end of the core probe and a sheath catheter enclosing the core probe and the coil. The coil has a first shape when constrained within the sheath and a second shape once it extended through a distal end of the sheath catheter. In various embodiments, the first shape is straight, the second shape is one or more of an apex vortex shape, a spiral shape, or a J-shape. The coil optionally includes a shape memory material, while the sheath optionally includes an insulating material and the device optionally includes a handle which houses a radiofrequency source. The device also optionally includes a mechanical component that is connected to the sheath catheter and permits manipulation of the position and/or length of the sheath catheter. The mechanical component may also or additionally permit manipulation of the position of the coil. In some embodiments, the ablation device is configured to be inserted through a working channel of an endoscope or ERCP device and/or to be visible by ultrasound.

In yet another aspect, the invention relates to methods of treating a patient which include inserting a biopsy needle into a lesion, deploying a coil electrode through the needle into the lesion, and delivering radiofrequency energy to the lesion, thereby heating or ablating the lesion and/or the surrounding tissue. In various embodiments, the biopsy needle is suitable for fine needle ablation, the cyst is a pancreatic cyst, the coil electrode comprises a wire and deploying the coil electrode comprises advancing about 5-10 inches of the length of the wire through the distal end of the needle into the cyst. The method may also include ultrasound guided endoscopic placement of the needle and/or aspiration of fluid or tissue from the lesion. In some cases, deployment of the coil electrode and delivery of radiofrequency to the lesion is based on an assessment of a predetermined characteristic, such as macroscopic characteristic associated with malignancy, a cytological characteristic associated with malignancy, a histological characteristic associated with malignancy, expression of a protein associated with malignancy, or the presence of a nucleic acid associated with malignancy. A characteristic “associated with malignancy” is, more generally, any characteristic that would be relied upon by a medical professional to make a definitive or tentative conclusion that a particular lesion is, or may become, malignant.

Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, routines, steps, or characteristics may be combined in any suitable manner in one or more examples of the technology. The terms “substantially” and “approximately” mean ±10% and, in some embodiments, within ±5%. The headings provided herein are for convenience only and are not intended to limit or interpret the scope or meaning of the claimed technology.

The phrase “and/or,” as used herein should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

This specification makes repeated reference to cysts, but it will be understood that these references are exemplary and are not necessarily limiting. For clarity, devices, systems and methods of the invention are useful in treating cysts as well as structures not delineated by a clear margin or membrane such as pseudocysts, abscesses and, more generally, any void or portion of a void or lumen within the body of a patient where treatment or ablation of tissue adjoining the void or lumen is desired.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which:

FIG. 1A is a cross sectional view of an endoscope;

FIGS. 1B and 1C schematically depict an ablation device integrated with the endoscope in accordance with various embodiments of the invention;

FIGS. 1D and 1E schematically depict an ablation device integrated with a surgical instrument in accordance with various embodiments of the invention;

FIGS. 2A-2C schematically depict the ablation device in accordance with various embodiments of the invention;

FIG. 3A schematically depicts the ablation device in accordance with an embodiment of the invention;

FIGS. 3B and 3C schematically depict operational mechanisms of the ablation device in accordance with various embodiments of the invention; and

FIGS. 4A-4E schematically depict exemplary shapes of a coil implemented in the ablation device in accordance with various embodiments of the invention;

### DETAILED DESCRIPTION

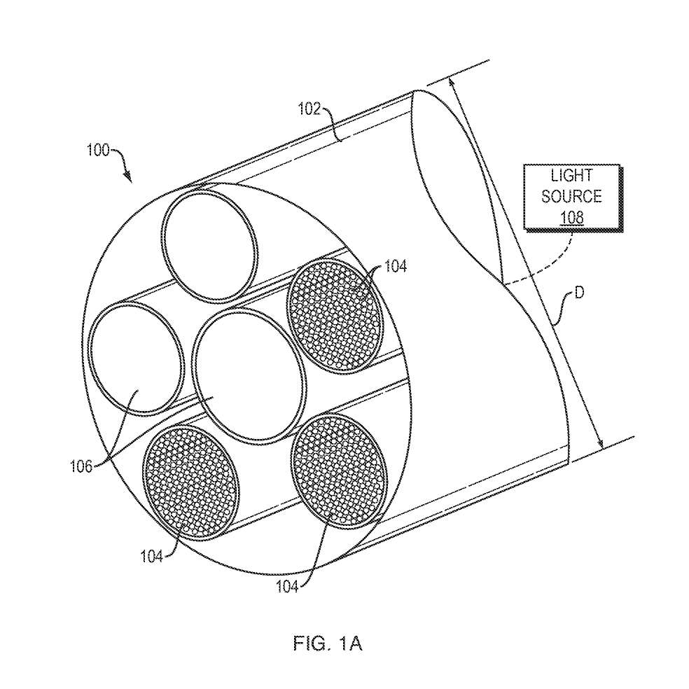

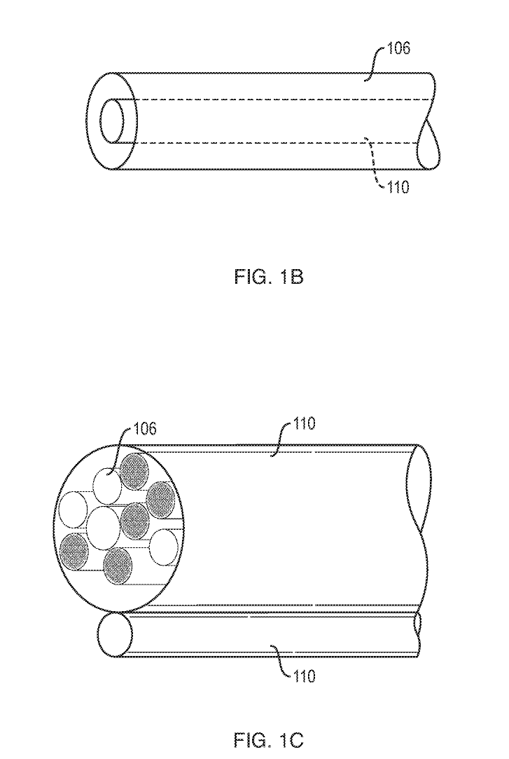

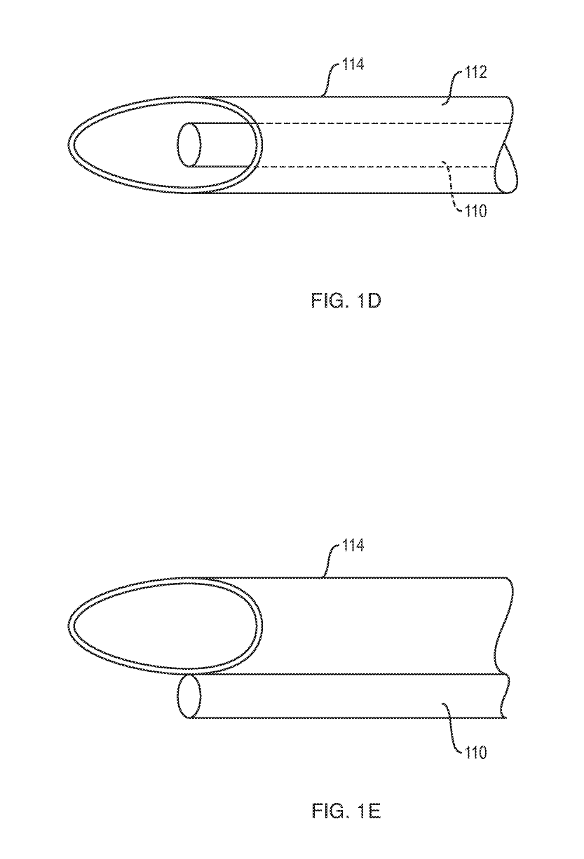

Refer first to FIG. 1A, which illustrates an endoscope 100 typically including a catheter 102 having optical fibers 104 and multiple long, narrow working channels 106; the overall outer diameter D ranging from 5 mm to 15 mm is constrained by the limited dimensions of the body cavity opening (e.g., throat, intestine, trachea). The working channels 106 may include air and/or water channels that provide an airtight or watertight internal compartment for components such as electrical wiring and controls to be integrated therein, thereby protecting the components from exposure to patient secretions during use as well as facilitating submersion of the endoscope for cleaning and subsequent disinfection. The endoscope 100 generally includes a light source (such as a light-emitting diode), a halogen light source, or a metal halide light source) 108 that emits light via the optical fibers 104 to the distal (insertion) end of the catheter 102. Referring to FIG. 1B, in various embodiments, a tumor ablation device 110 is inserted into at least one working channel 106 to facilitate ablation of tumor tissue (such as a tumor cyst) at a target site. Alternatively, referring to FIG. 1C, the tumor ablation device 110 may be attached to or joined to the endoscope 100 to form a single device.

Referring to FIG. 1D, in various embodiments, the tumor ablation device 110 is disposed within and/or capable of being deployed from a lumen 112 of a surgical instrument 114, such as an FNA needle. A surgical instrument 114 suitable for use with the tumor ablation device may have a gauge (“ga”) suitable for use in percutaneous or endoscopic needle aspiration, for example 25 ga, 23 ga, 22 ga, 19 ga, etc. The tumor ablation 110 device is preferably configured to fill part or all of the volume and shape of a cyst or other void for which ablation is desired. For example, the volume may be about 1 cubic centimeter (“cc”) and may be, for example, substantially spherical, oblate, discoid, ovoid, ellipsoid, or irregular. Again, the tumor ablation device may be attached to or joined to the surgical instrument 114 to form an integrated single device as depicted in FIG. 1E. Utilization of the ablation device 110 with the surgical instrument 114 enables the physician to treat the tumor cyst immediately after, for example, draining the cyst, if desired.

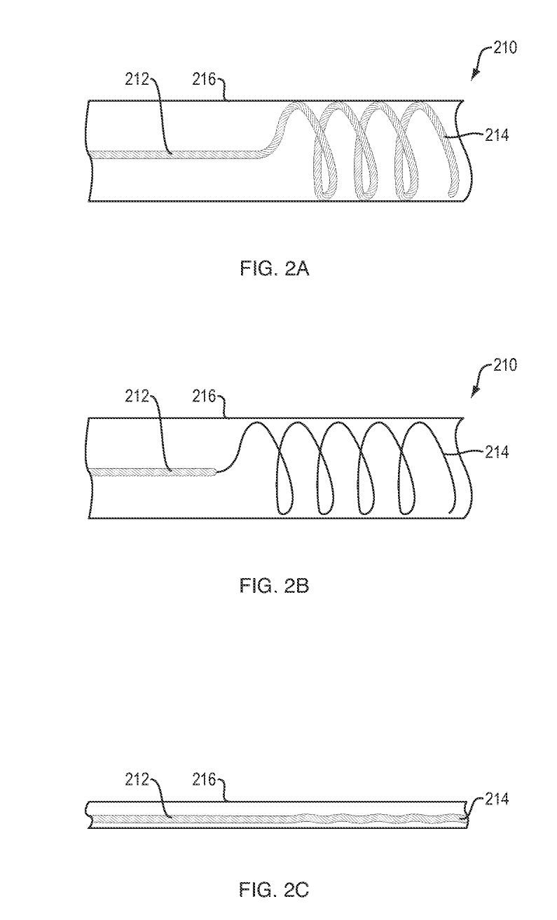

FIGS. 2A-2C depict various exemplary tumor ablation devices 210 in accordance with various embodiments of the current invention. The ablation device 210 may include a central core (or a radiofrequency probe) 212 and a coil 214 at the distal end thereof. The coil 214 may be an extension of the central core 212 (FIG. 2A) or may attach to the central core 212 using, for example, a weld (FIG. 2B). It should be noted that the terms “distal” and “proximal,” as used herein, are intended to refer to a direction away from (distal) and towards (proximal) a user of the device. In some embodiments, the central core 212 is preferably made of a conductive material which is stiff enough to push the coil 214 out of the distal end of the ablation device 210 but flexible enough to enable the physician to guide the coil 214 to the target region.

The coil 214, in preferred embodiments, includes one or more metals or metal alloys, such as platinum, a platinum alloy (e.g., platinum-tungsten alloy), or stainless steel. In a preferable embodiment, the coil 214 is made of a shape-memory material, such as Nitinol. Because a shape-memory material “remembers” its original, cold-forged shape and can be deformed substantially and still return to that shape, one of the advantages of using a shape-memory material is the high level of recoverable plastic strain that can be included. The maximum recoverable strain the shape-memory material can hold without permanent damage may be, for example, 8%, much larger than conventional steels with a maximum strain of, e.g., 0.5%. Therefore, the shape-memory material can be used to provide the coil 214 with a permanent shape, which the coil 214 assumes when in an unstressed configuration. This advantageously permits the coil 214 to be drawn into the surgical instrument prior to deployment, and to be bent, curved, etc. as the surgical instrument

The coil 214 is generally made from a narrow gauge (e.g. 1/1000″- 1/100″ or 0.00254 cm-0.0254 cm) wire with a length of between 5 and 10 inches (12-25 cm), so that, when the coil 214 is deployed, the cyst is filled with a length of coil that is sufficient to achieve contact with or proximity to substantially of the wall of the cyst to be treated. Those of skill of art will appreciate that a substantial length of coil is used to fill or partially fill the volume of a cyst. For example, to fill a volume of about 1 cc, a length of wire ranging between 5 and 10, 10 and 15, 15 and 20 and 20 or more cm may be inserted into the cyst. In various embodiments, the wire forming the coil 214 has a length of 5 cm, 10 cm, 15 cm, 20 cm, 25 cm, 30 cm or more. One or more of the ablation device 210, endoscope or ECRP device 100 or needle 114 preferably has a length sufficient to accommodate the full retracted length of the wire coil 214 and the central core 212. As explained below, the wire may remain coiled when it is retracted into the ablation device 210 or it may be straightened out. In some instances, when the coil is retracted the wire is straightened in one portion of the ablation device 210 while it is coiled in another portion of the ablation device 210.

In various embodiments, the ablation device 210 further includes a sheath catheter 216 to enclose the central core 212 and the coil 214 therewithin. The sheath catheter 216 is preferably made of an insulating material. Additionally, the sheath catheter 216 may have a very small outer diameter (e.g., ˜2 millimeters) such that the entire ablation device 210 is insertable into the working channel 106 of the endoscope 100 or the lumen 112 of the surgical instrument 114. The coil 214 may be constrained in a coiled form within the sheath catheter 216 as depicted in FIGS. 2A and 2B. Alternatively, the coil 214 may be straightened in the sheath catheter 216 for shipping (FIG. 2C) and furled into the coiled form upon exiting the sheath catheter 216 or being deployed in the target tumor tissue. Delivering the coil 214 using an unfurled form in the sheath catheter 216 advantageously reduces the required outer diameter of the sheath catheter 216, thereby allowing the ablation device 210 to be easily inserted into the working channel 106 of the endoscope 100 or the, lumen 112 of the surgical instrument 114. Accordingly, the coil 214 may have a temporary shape and a permanent shape: The coil 214 exhibits its temporary shape when being constrained within the sheath catheter 216. As the coil 214 exits the sheath catheter 216, the coil 214 is released from the temporary shape and exhibits its permanent shape. The temporary shape and permanent shape may be the same or different.

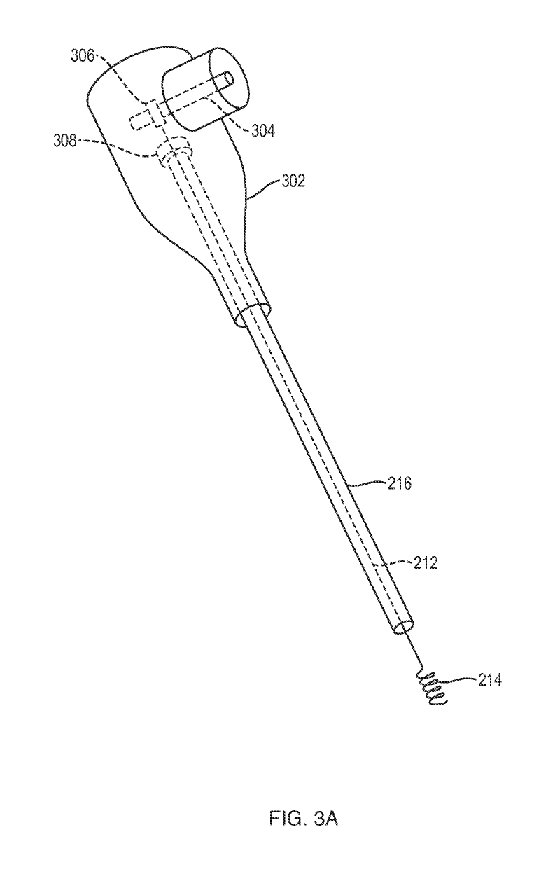

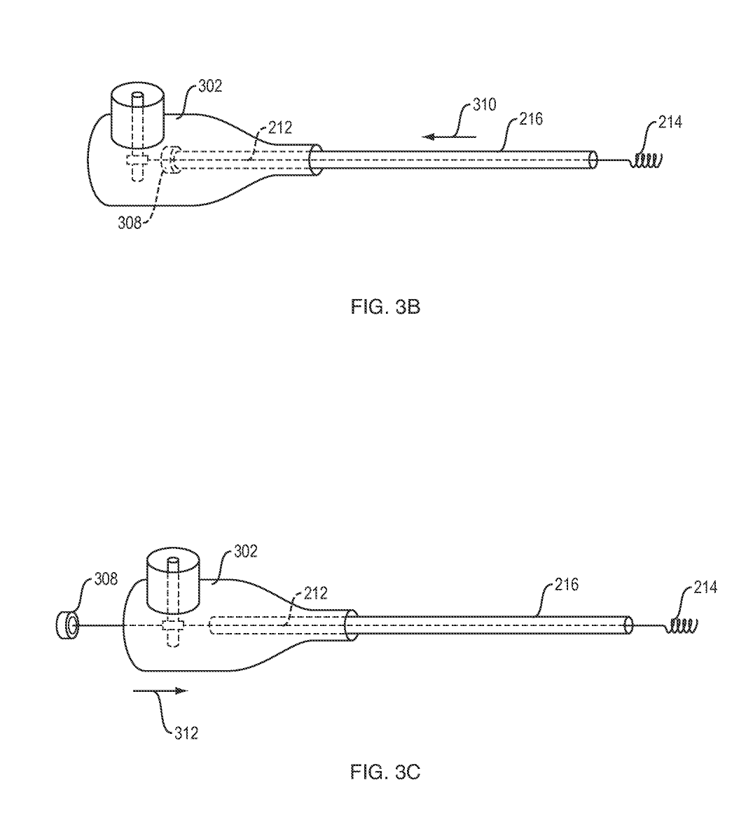

Referring to FIGS. 3A-3C, in some embodiments, the sheath catheter 216 interfaces with a handle 302 that, in turn, connects to a radiofrequency source 304 such that the proximal end of the central core 212 is electrically connected to the radiofrequency source 304 via, for example, an electrical connector 306. This therefore allows the radiofrequency source 304 to directly deliver ablation energy to the coil 214 for treating the target tumor cyst. The radiofrequency energy delivered to the cyst is sufficient to ablate the tumor tissue and eliminate or slow down the growth thereof. Alternatively, the radiofrequency energy is sufficient only to heat the tissue without ablating it. The handle 302 may also include a mechanical component 308 for facilitating deployment of the coil 214. In one implementation, the mechanical component 308 is connected to the sheath catheter 216 for controlling the position and/or length thereof. For example, the component 308 may retract the sheath catheter 216 in a direction 310 to a positive stop (not shown) located at the proximal end, thereby exposing the coil 214 to the target cyst as depicted in FIG. 3B. In another implementation, the mechanical component 308 is connected to the central core 212 for manipulating the position of the coil 214. For example, the component 308 may push the central core 212 in a direction 312 passing the sheath catheter 216 (that may have a fixed position) to the distal end, thereby exposing the coil 214 as depicted in FIG. 3C. The mechanical component 308 may be located inside or outside the handle 302. In addition, exposing the coil 214 outside the delivery sheath catheter 216 using the approaches as described above are exemplary embodiments, any other means that is suitable for exposing the coil 214 to the target tumor tissue is within the scope of the current invention. In one embodiment, the handle 302 is sized and shaped to be grasped by the physician.

During operation, an operator (e.g. a physician) first gains access to the cyst to make a diagnosis using, for example, an endoscope or ERCP device, and/or perform a procedure, such as drainage or biopsy, using a surgical instrument (e.g., an FNA needle). If an immediate treatment is necessary, the physician may insert the ablation device 210 into the lumen 112 of the FNA needle 114 or the working channel 106 of the endoscope 100 and deploy the coil 214 within the cyst. The physician may then activate the radiofrequency source 304 to deliver sufficient energy to the coil 214 for treating the tumor cyst. In various embodiments, the ablation device 210 is integrated with the endoscope or surgical instrument and form a single device. The physician may perform the tumor treatment simply by deploying the coil 114 into the tumor cyst and subsequently switching on the radiofrequency source 304 to deliver ablation energy to the target cyst. When a treatment goal is achieved (i.e., ablating at least a portion of tumor tissue or eliminating or slowing down the growth thereof), the physician may determine to stop the treatment. In one embodiment, the coil 214 is retracted to the sheath catheter 216 using the mechanical component 308 in a manner as described above and re-constrained within the sheath catheter 216. The ablation device 210 (and the endoscope 100 or surgical instrument 114) may then be removed from the patient's body.

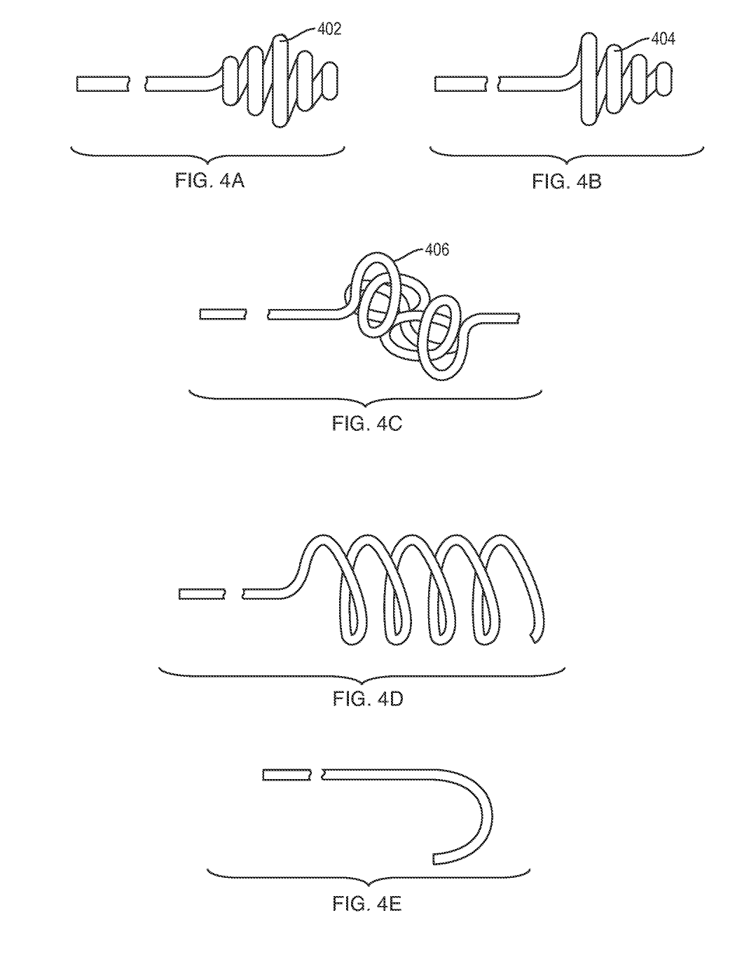

The coil 214 may have various permanent shapes depending on the structure of the target cyst. In a preferable embodiment, the permanent shape is capable of “packing” the space of the cyst's cavity such that the coil 214 is in contact with the maximum possible surface area thereof. This ensures that the cyst can be efficiently and effectively ablated or treated using the deployed coil 204. Referring to FIGS. 4A-4D, the coil 214 may have a dual apex vortex shape (or “ball” shape), a single apex vortex shape (or “cone” shape), a complex shape (or “bird's nest” shape), or a spiral shape as depicted in FIGS. 4A. 4B, 4C, and 4D, respectively. In one embodiment, referring to FIG. 4E, the coil 214 has a permanent shape in the form of a “J,” which may be used, for example, to fill remaining space in the cavity that was not filled by other coils. For example, the physician may hook the curved portion of the J coil into a coil that has been deployed within the cyst and then shape the straighter portion of the J coil to fill the space within the cavity. The shape of the coil 214 is not limited to the above-identified shapes; for example, the coil 214 may have a triangular, rectangular, octagonal or other shape. Any shape that is suitable for being inserted into a tumor cyst for ablation purposes is within the scope of the current invention. In addition, the coil 214 may include any number of different shapes, which may, again, depend on the structure of the cyst.

Finally, implants utilizing coil electrodes according to the present invention may be used in bipolar or monopolar configurations, as will be appreciated by those of skill in the art.

Certain embodiments of the present invention were described above. It is, however, expressly noted that the present invention is not limited to those embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

What is claimed is:

1. A method of treating a patient, comprising:
inserting a distal tip of a biopsy needle into a lesion in the body of the patient;
deploying, through the distal tip of the biopsy needle, a coil electrode within the lesion; and
delivering radiofrequency to the lesion.

2. The method of claim 1, further comprising the step of aspirating a fluid or a tissue from the lesion.

3. The method of claim 1, wherein the biopsy needle is configured for fine needle ablation.

4. The method of claim 1, wherein the lesion is a pancreatic cyst.

5. The method of claim 1, wherein a the coil electrode comprises a wire, and wherein deploying the coil electrode comprises advancing a length of between about 5 inches and about 10 inches of the wire through a distal end of the biopsy needle into the cyst.

6. The method of claim 1, wherein the step of inserting the distal tip of the biopsy needle comprises delivering the biopsy needle to the lesion through an endoscope and identifying a location of the lesion by ultrasound.

7. The method of claim 1, wherein the coil electrode contacts a wall of the lesion when deployed.

8. A method of treating a patient, comprising:
inserting a distal tip of an aspiration needle having a gauge between about 18 and about 25 into a pancreatic cyst;
aspirating a fluid or a tissue from the pancreatic cyst; and
based on the assessment of a predetermined characteristic within the fluid or tissue, deploying a coil electrode through the distal tip of the aspiration needle and delivering, via the coil electrode, radiofrequency energy to the pancreatic cyst.

9. The method of claim 8, wherein the predetermined characteristic is selected from the group consisting of a macroscopic characteristic associated with malignancy, a cytological characteristic associated with malignancy, a histological characteristic associated with malignancy, expression of a protein associated with malignancy, and the presence of a nucleic acid associated with malignancy.

10. A system for treating a patient, comprising:
a needle sized to fit within a working channel of an endoscope; and
a wire electrode disposed at least partially within a lumen of the needle comprising a shape memory material and configured to be straightened when disposed within the lumen and to assume a coiled shape when outside of the needle,
wherein the wire electrode comprises a wire having a diameter between 1/1000 and 1/100 inch, a length of between 5 and 10 inches, and defines a volume of about 1 cc when coiled.

11. The system of claim 10, wherein the coiled shape is selected from the group consisting of spherical, oblate, discoid, ovoid, ellipsoid, irregular, and J-shaped.

12. The system of claim 10, wherein the needle has a gauge of between about 18 and about 25.

13. The system of claim 10, wherein the wire electrode includes a shape memory material.

14. The system of claim 10, further comprising a source of radiofrequency energy electrically coupled to the wire electrode.

15. The system of claim 10, further comprising a handle configured to advance and retract the wire electrode through the needle.

16. A method of treating a patient utilizing the system of claim 10, the method comprising:
inserting a distal portion of the needle into a body of the patient;
aspirating a fluid from the body of the patient through the needle;
advancing at least a portion of the wire electrode through the distal portion of the needle and into the body of the patient, such that the wire electrode contacts a tissue surface; and
delivering, through the wire electrode, radiofrequency energy to the tissue surface, thereby heating the tissue surface.

17. The method of claim 16, wherein the biopsy needle is configured for fine needle ablation.

18. The method of claim 16, wherein the step of inserting the distal tip of the needle into the body includes identifying a lesion by ultrasound, and advancing the biopsy through an endoscope and to the lesion.

19. The method of claim 18, wherein the lesion is a pancreatic cyst.

20. The method of claim 19, wherein the step of advancing the wire electrode comprising placing at least a portion of the wire electrode within the pancreatic cyst in contact with or close proximity to the wall of the pancreatic cyst.
